(19) United States
(12) Patent Application Publication
(10) Pub. No.: US 2023/0270503 A1
(43) Pub. Date: Aug. 31, 2023

(54) SEGEMENTAL TRACKING COMBINING OPTICAL TRACKING AND INERTIAL MEASUREMENTS

(71) Applicant: Mazor Robotics Ltd., Caesarea (IL)

(72) Inventors: Eliyahu Zehavi, Tel Aviv (IL); Yonatan Ushpizin, Glil Yam (IL); Ido Zucker, Tel Aviv (IL); Avraham Turgeman, Beer Yaakov (IL); Elad Rotman, Netanya (IL); Adi Ess, Ramat Gan (IL)

(21) Appl. No.: 17/591,717

(22) Filed: Feb. 3, 2022

(51) Int. Cl. A61B 34/20; A61B 34/30; G06T 7/73

(52) U.S. Cl. CPC A61B 34/20; A61B 34/30; G06T 7/73; A61B 2034/2048; A61B 2034/2057; A61B 2034/2065; A61B 2034/2068; G06T 2207/30204

(57) ABSTRACT

A system according to at least one embodiment of the present disclosure includes a processor; and a memory storing instructions thereon that, when executed by the processor, cause the processor to: receive, from an inertial sensor disposed proximate an anatomical element, a reading indicative of a first movement of the anatomical element; determine a second movement of a fiducial marker being positioned with a known physical relationship to the inertial sensor; and determine, based on the first movement and the second movement, a change in pose of the anatomical element.

### BACKGROUND

The present disclosure is generally directed to segmental tracking and relates more particularly to tracking anatomical elements using optical tracking and inertial measurements.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A system according to at least one embodiment of the present disclosure comprises: a processor; and a memory storing data thereon that, when executed by the processor, cause the processor to: receive, from an inertial sensor disposed proximate an anatomical element, a reading indicative of a first movement of the anatomical element; determine a second movement of a fiducial marker being positioned with a known physical relationship to the inertial sensor; and determine, based on the first movement and the second movement, a change in pose of the anatomical element.

Any of the aspects herein, wherein the inertial sensor comprises an inertial measurement sensor (IMU) disposed on the anatomical element.

Any of the aspects herein, wherein the fiducial marker is disposed on the IMU.

Any of the aspects herein, wherein the anatomical element comprises a vertebra.

Any of the aspects herein, wherein the inertial sensor comprises at least one of a gyroscope or an accelerometer.

Any of the aspects herein, wherein the fiducial marker comprises an optical sphere.

Any of the aspects herein, wherein the first movement comprises a translational movement, wherein the second movement comprises a rotational movement about a first axis associated with the anatomical element, and wherein the change in pose of the anatomical element is determined to include at least some of the translational movement and at least some of the rotational movement about the first axis.

Any of the aspects herein, wherein an imaging device captures the second movement of the fiducial marker.

Any of the aspects herein, wherein the data further cause the processor to: maneuver a robotic arm to move based on the determined change in pose of the anatomical element.

A method according to at least one embodiment of the present disclosure for tracking a movement of an anatomical element comprises: receiving, from a first inertial sensor attached to the anatomical element, first information indicative of the movement of the anatomical element; receiving, from a first imaging device, second information indicative of a second movement of a first tracking marker; and determining, based on the first information and the second information, a change in pose of the anatomical element.

Any of the aspects herein, further comprising receiving, from a second inertial sensor attached to a second anatomical element, third information indicative of a third movement of the second anatomical element; receiving, from a second tracking marker, fourth information indicative of a fourth movement of a second tracking marker; and determining, based on the third information and the fourth information, a change in pose of the second anatomical element.

Any of the aspects herein, further comprising: controlling a surgical tool based on at least one of the change in pose of the anatomical element or the change in pose of the second anatomical element.

Any of the aspects herein, further comprising: updating, based on at least one of the change in pose of the anatomical element or the change in pose of the second anatomical element, a surgical plan.

Any of the aspects herein, wherein the first tracking marker is an optical sphere.

Any of the aspects herein, wherein the optical sphere is tracked by the first imaging device.

Any of the aspects herein, wherein the first tracking marker comprises an Infrared Light Emitting Diode (IRED).

Any of the aspects herein, further comprising: registering a robotic arm to the anatomical element.

Any of the aspects herein, wherein the movement is a translational movement, and wherein the second movement is a rotational movement about a first axis of the anatomical element.

A system according to at least one embodiment of the present disclosure comprises: an imaging device; an inertial measurement unit (IMU) disposed on an anatomical element; at least one fiducial marker disposed on or in proximity to the IMU; a processor; and a memory storing data thereon that, when processed by the processor, cause the processor to: receive, from the IMU, information describing a translational movement of the anatomical element; receive, from the imaging device, information describing a rotational movement of the at least one fiducial marker; and determine, based on the information describing the translational movement and the information describing the rotational movement, a pose of the anatomical element.

Any of the aspects herein, wherein the fiducial marker comprises an optical sphere, and wherein the IMU comprises at least one of an accelerometer or a gyroscope.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a block diagram of tracking elements attached to an anatomical element according to at least one embodiment of the present disclosure;

FIG. 2B is a block diagram of tracking elements and an anatomical element according to at least one embodiment of the present disclosure;

FIG. 2C is a block diagram of a tracking element disposed on multiple anatomical elements according to at least one embodiment of the present disclosure;

FIG. 3A is a block diagram of tracking elements attached to anatomical elements according to at least one embodiment of the present disclosure;

FIG. 3B is a block diagram of an anatomical element moving relative to another anatomical element according to at least one embodiment of the present disclosure;

FIG. 3C is a block diagram of the anatomical elements after the movement of at least one anatomical element according to at least one embodiment of the present disclosure; and

FIG. 4 is a flowchart according to at least one embodiment of the present disclosure; and

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In navigation- or robotic-aided spine surgeries, one challenge may be keeping the system registered to the anatomy of the patient. According to at least one embodiment of the present disclosure, a method to address this challenge may be to place an optical instrument or other marker on the anatomy of the patient (e.g., a vertebra of the spine) which can be linked to or tracked by the navigation system. In some embodiments, the method may include registering the spine (or portions thereof) to the system based on the optical instrument attached to the vertebra. However, the registering of the spine in such embodiments may be based on the single optical instrument fixed on a vertebra of the spine, leading to a possible decrease in accuracy of the registration. Another possible method to address the foregoing challenges may be to place optical instruments (e.g., LEDs, reflective markers, etc.) on each vertebra or other anatomical elements of the patient and track each vertebra individually. However, difficulties associated with tracking the optical instruments for multiple anatomical elements (e.g., visibility, distinguishing between each optical instrument and/or each anatomical element, etc.) may unnecessarily complicate the surgery or surgical procedure (e.g., extra time, processing power, etc. must be spent identifying each optical instrument). To address these issues and others, embodiments of the present disclosure may combine the use of optical instruments and IMUs to track the anatomical elements (e.g., vertebrae) during a surgery or surgical procedure.

It may be beneficial to obtain the movement of the anatomical elements in the six degrees of freedom afforded by 3D space to help remedy the decreased accuracy. In one method of the present disclosure, the surgical tool used to operate on the anatomical elements may have three markers attached thereto. The use of the three markers may enable unique physical structures that may be subject to geometric constraints (e.g., the distance between each marker is greater than 40 millimeters (mm)). While such a method can permit a single vertebra to be tracked as the surgical tool operates, when multiple vertebra are to be tracked, alternative methods may be implemented.

In at least one embodiment of the present disclosure, the above challenges may be addressed by combining optical tracking with IMU sensors, with each combination disposed on each vertebra of the spine. The spheres or any other optical instrument can be tracked (e.g., using imaging devices, navigation systems, etc.) to determine three-dimensional (3D) movements of each vertebra, while measurements, readings, or other data generated by the IMU sensors can be used to determine 3D rotations. The combination of the spheres and the IMU sensors may enable anatomical element movement detection for each individual anatomical element with minimum interruption to the surgical procedure. In some embodiments, the robotic arms used to maneuver the surgical tool may be combined with the optical tracking in a single coordinate system (e.g., to facilitate registration).

Embodiments of the present disclosure provide technical solutions to one or more of the problems of (1) tracking multiple vertebrae of the spine during a surgery or surgical procedure; and (2) registration issues associated with tracking the pose of the spine using one vertebra.

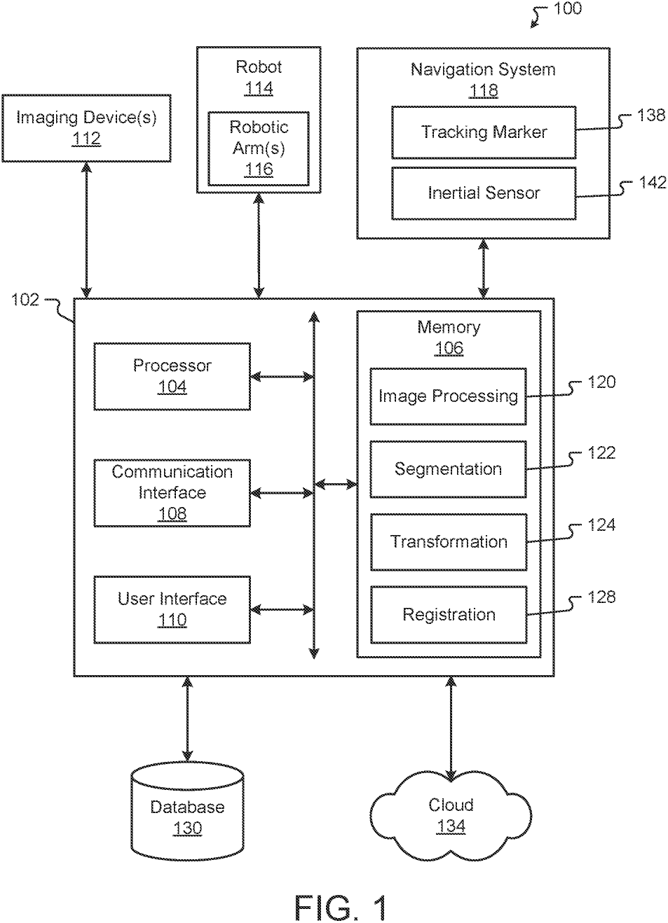

Turning first to FIG. 1, a block diagram of a system 100 according to at least one embodiment of the present disclosure is shown. The system 100 may be used to track one or more optical trackers positioned on or near one or more anatomical elements; to register the one or more anatomical elements to a navigation system; to control, maneuver, and/or otherwise manipulate a surgical mount system, a surgical arm, and/or surgical tools attached thereto based on the registration and/or the optical trackers; and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.1 1a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and/or orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112, robotic arm 116, surgical tools) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). In some embodiments, the navigation system 118 may use the imaging device 112 and/or data captured using the imaging device 112 to track the reference markers, navigated trackers, or other objects within the operating room. The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The navigation system 118 may comprise a one or more tracking markers 138. The tracking markers 138 may assist the navigation system 118 in determining one or more poses (e.g., positions and/or orientations) of one or more anatomical elements (e.g., vertebrae, ribs, soft tissues). The tracking markers 138 may be disposed on or proximate to one or more anatomical elements. In some embodiments, the tracking markers 138 may be positioned in other areas in the surgical environment (e.g., on other portions of the patient a known physical distance from the one or more anatomical elements, on or proximate one or more imaging devices 112, on or proximate one or more robotic arms 116, combinations thereof, and/or the like). The number and/or density of the number of tracking markers 138 disposed on, proximate to, or otherwise used to identify the one or more anatomical elements may be changed, altered, or otherwise chosen depending upon, for example, the type of anatomical element, the type of surgery or surgical procedure, combinations thereof, and/or the like.

The tracking markers 138 may be or comprise optical components (e.g., elements that provide visual indicia) that may assist the navigation system 118 in determining a location of each of the tracking markers 138 within the surgical environment (e.g., relative to other tracking markers, relative to one or more anatomical elements, relative to other components of the system 100, combinations thereof, and/or the like). For instance, the tracking markers 138 may each be reflective, luminescent, or otherwise provide a visual indicator capable of being captured by the navigation system 118 (e.g., using the imaging device 112) to determine the pose of the tracking markers 138. In some embodiments, the tracking markers 138 may include light emitting diodes (LEDs) and/or infrared light emitting diodes (IREDs) that emit visible light or other forms of electromagnetic radiation at various frequencies. In at least one embodiment, the tracking markers 138 may comprise optical spheres (e.g., reflective spheres with a 1 millimeter (mm), 2 mm, 3 mm, 4 mm, 5 mm, 6 mm, 7 mm, 8 mm, 9 mm, or 10 mm radius, or spheres with smaller or larger radii). The optical sphere size may be based on, for example, the type of anatomical element on which or proximate to which the sphere is placed, the type of surgery or surgical procedure, combinations thereof, and/or the like.

In some embodiments, the tracking markers 138 may passively and/or actively generate indicia to assist the navigation system 118 in identifying the tracking markers 138. For instance, the tracking markers 138 with LEDs and/or IREDs may be wired or wirelessly connected to a controller, processor, or other computing device (e.g., a computing device 102) that generate and send signals that selectively illuminate the tracking markers 138. The signals may cause the tracking markers 138 to provide indicia at various frequencies, pulse rates/duty cycles, and/or intensities (e.g., color intensity, brightness intensity). In some embodiments, the tracking markers 138 may be illuminated based on a surgical plan, the type of surgery or surgical procedure, the requirements of the navigation system 118 (e.g., the illumination occurs when the navigation system 118 or a user like a surgeon determines that the pose of one or more tracking markers 138 has not been determined), combinations thereof, and/or the like.

The navigation system 118 may comprise one or more inertial sensors 142. The inertial sensors 142 measure forces to, changes in angular momentum of, and/or changes in orientation (e.g., changes in pitch, yaw, and/or roll) of itself or of a component (e.g., an anatomical element) to which the inertial sensor 142 is attached. For instance, the inertial sensor 142 may measure the rotation or other movement of an object (e.g., an anatomical element) to which the inertial sensor 142 is attached when the object moves (e.g., when the object rotates, when the object experiences a force). In some embodiments, the inertial sensor 142 may be or comprise an inertial measurement unit (IMU). The IMU may be or comprise accelerometers, gyroscopes, magnetometers, combinations thereof, and/or other components for detecting the movement of the inertial sensor 142. The inertial sensors 142 may be positioned on or a known physical distance from one or more anatomical elements (e.g., vertebrae, ribs). As such, the movement of the inertial sensors 142 may be converted or transformed into an associated movement of the object to which the inertial sensors 142 is attached or to an object proximate the inertial sensors 142 based on the physical relationship between the inertial sensors 142 and the object. For instance, an inertial sensor 142 may be disposed on a vertebra that rotates in a first direction about a first axis. The measurement of the inertial sensors 142 measuring the rotation may be converted into a respective rotation of the vertebra based on the physical relationship between the inertial sensor 142 and the vertebra (e.g. the inertial sensor 142 is mounted on the end of an elongated rod extending out of the vertebra).

In some embodiments, the inertial sensors 142 may be connected to or coupled with the tracking markers 138. For instance, an inertial sensor 142 may be disposed within a tracking marker 138 (e.g., the tracking marker 138 comprises an optical sphere that includes a hollow cavity, with the inertial sensor 142 disposed within the hollow cavity). The combination of the inertial sensors 142 and the tracking markers 138 may allow for a compact device to be disposed on or proximate to one or more anatomical elements to enable tracking any pose changes in the one or more anatomical elements during the course of a surgery or surgical procedure.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient’s anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of the method 400 described herein. The system 100 or similar systems may also be used for other purposes.

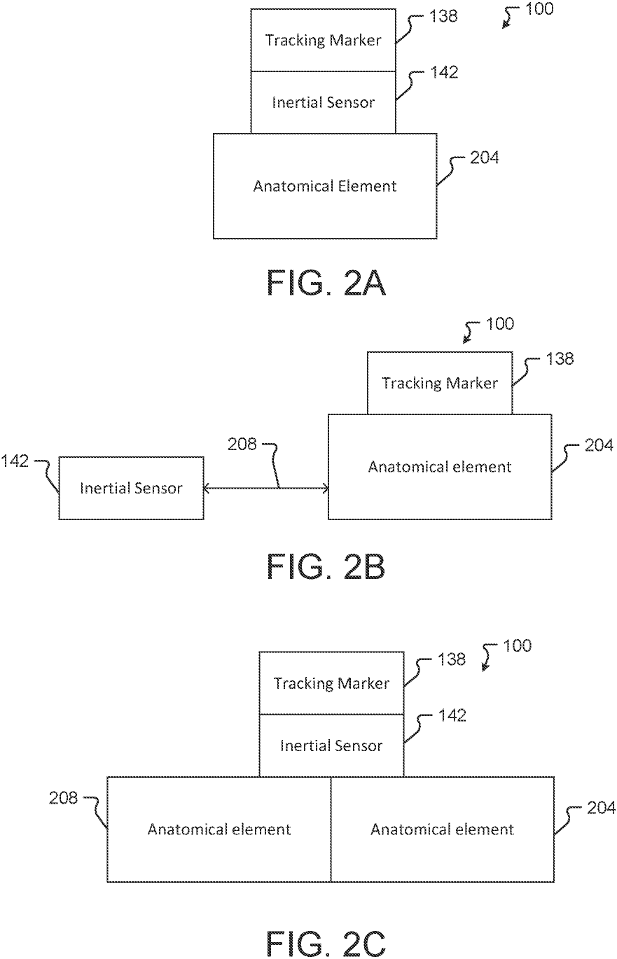

FIGS. 2A-2C illustrate aspects of the system 100 in accordance with at least one embodiment of the present disclosure. As discussed herein, the system 100 may comprise one or more tracking markers 138 and one or more inertial sensors 142 that may be disposed a known physical distance from or in a known physical relationship with one or more anatomical elements 204. The anatomical elements 204 may be or comprise organs, bones, portions thereof, or any portion of a human anatomy (e.g., a spinal column, a vertebrae, etc.). The anatomical elements 204 may be portions of a patient upon which a surgery or surgical procedure is to be conducted (e.g., a vertebra upon which a vertebral fusion is to be performed, a vertebra to be drilled into to relieve pressure on a nerve). The number and type of the anatomical elements 204 may vary depending on, for example, the type of surgery or surgical procedure being performed. For instance, in some embodiments the anatomical elements 204 may comprise one or more vertebra of the spine. In some embodiments, different spinal surgeries or surgical procedures may use or require the tracking markers 138 and/or the inertial sensors 142 being attached and/or disposed proximate different vertebrae. For example, a spinal fusion between the T6 and the T7 vertebrae may include placing additional tracking markers 138 and/or inertial sensors 142 on or proximate the T6 and T7 vertebrae, while a different spinal procedure on the T2 vertebra may have additional tracking markers 138 and/or inertial sensors 142 positioned on the T1 and T3 vertebrae to track movement of the T2 vertebra during the surgical procedure.

In embodiments where the tracking markers 138 and the inertial sensors 142 are each paired up in a combined apparatus, one or more of the apparatuses may be affixed or attached to one or more of the anatomical elements 204. In other words, each of the inertial sensors 142 may be disposed in each of the tracking markers 138 (such as when the tracking markers 138 include optical spheres into which the inertial sensors 142 are deposited), and the tracking markers 138 may be attached to one or more of the anatomical elements 204.

Additionally or alternatively, one or more of the inertial sensors 142 may have a physical relationship with the anatomical element 204 and/or the tracking markers 138, as shown in FIG. 2B. Stated differently, the inertial sensor 142 may not be directly attached to the anatomical element 204 and/or the tracking markers 138 and may instead be positioned a first distance 208 from the anatomical element 204 and/or any one of the tracking markers 138. The value of the first distance 208 is in no way limited and may be, for example, 0.1 mm, 0.2 mm, 0.5 mm, 1 mm, 2 mm, 5 mm, 10 mm, 15 mm, 25 mm, 50 mm, 100 mm, 150 mm, 200 mm, or 500 mm from the anatomical element 204 and/or any one of the tracking markers 138.

In some embodiments, such as the embodiment depicted in FIG. 2C, the one or more tracking markers 138 and/or the one or more inertial sensors 142 may span or be connected across two or more anatomical elements (e.g., the anatomical element 204 and/or another anatomical element 206). For instance, the anatomical elements 204, 206 may be or comprise vertebrae, and an inertial sensor 142 may be disposed such that movement of the anatomical element 204 and/or the anatomical element 206 may be captured by the inertial sensor 142. Additionally or alternatively, the one or more tracking markers 138 may be positioned across the anatomical element 204 and/or the anatomical element 206 such that any movement of either anatomical element may result in the movement of the one or more tracking markers 138. While FIG. 2C depicts two anatomical elements, it is to be understood that additional anatomical elements may be tracked by or coupled together by the one or more inertial sensors 142 and/or the one or more tracking markers 138.

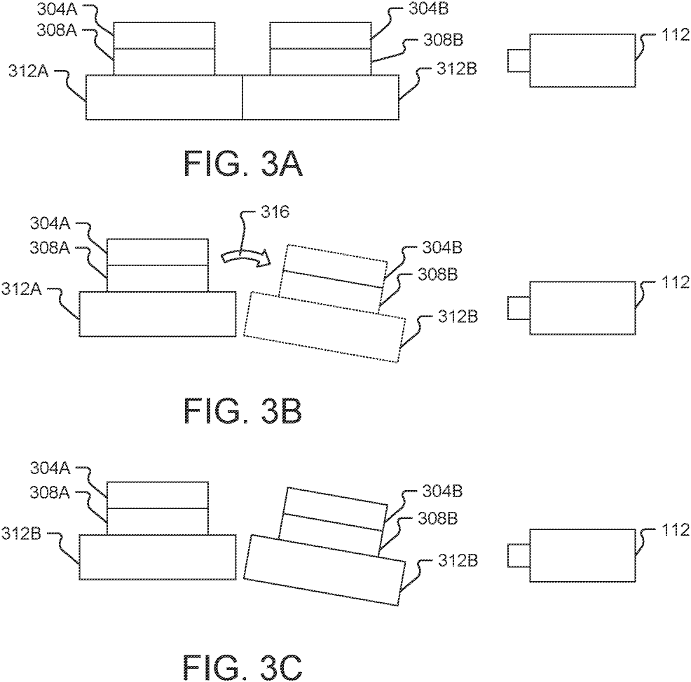

FIGS. 3A-3C depict aspects of the system 100 in accordance with at least one embodiment of the present disclosure. The aspects may comprise one or more anatomical elements 312A-312B. While FIGS. 3A-3C depict an anatomical element 312A and an anatomical element 312B, it is to be understood that additional or alternative anatomical elements may be present. Each of the anatomical elements 312A-312B may include one or more inertial sensors 308A-308B and/or one or more tracking markers 304A-304B. The anatomical elements 312A-312B may be similar to or the same as the anatomical element 204. The one or more inertial sensors 308A-308B may be similar to or the same as inertial sensors 142, and the one or more tracking markers 304A-304B may be similar to or the same as tracking markers 138. In some embodiments, the one or more imaging devices 112 may capture image data of the anatomical elements 312A-312B, the inertial sensors 308A-308B, and/or the tracking markers 304A-304B. The imaging devices 112 may pass the image data to one or more components of the system 100 (such as the navigation system 118). The navigation system 118 may use the image data for the purposes of, for example, operating or moving a surgical tool based on the image data.

In some embodiments, during the course of a surgery or surgical procedure, one or more of the anatomical elements 312A-312B may move. For instance, the anatomical element 312B may experience a movement 316 relative to the anatomical element 312A. The movement 316 may be caused by, for example, forces and/or vibrations caused by the operation of a surgical tool; forces generated by movement of another anatomical element; movement of a surgical bed upon which the patient is resting or the movement of any other surgical component; combinations thereof; and/or the like.

The movement 316 of the anatomical element 312B may be captured by the imaging devices 112, which may capture the movement of the tracking marker 304B and/or the inertial sensor 308B relative to, for example, the tracking marker 304A, the inertial sensor 308A, and/or the anatomical element 312A. The movement of the anatomical element 312B may be a translational movement (e.g., the anatomical element 312B moves relative to the anatomical element 312A in a first direction along a first axis), rotational movement (e.g., the anatomical element 312B rotates relative to the anatomical element 312A about a first internal axis, the anatomical element 312B rotates relative to the anatomical element 312A about a first axis of the anatomical element 312A), combinations thereof, and/or the like. The captured movement may be used by the navigation system 118 (or other component of the system 100 such as the computing device 102) to determine a new pose of the anatomical element 312B and adjust the surgery or surgical procedure accordingly.

As an example, the anatomical elements 312A-312B may be or comprise vertebrae, with a spinal surgery or surgical procedure being performed thereon. During the course of the spinal fusion, the navigation system 118 may navigate or otherwise operate a surgical tool (e.g., a drill) held by the robotic arm 116. As the surgical tool drills into the anatomical element 312A, the anatomical element 312B may experience the movement 316 relative to the anatomical element 312A (e.g., the torque generated by the surgical tool may generate a force to cause the anatomical element 312B to move). The movement 316 of the anatomical element 312B may, in some surgeries, negatively impact the surgery or surgical procedure (e.g., the vertebrae are no longer aligned to conduct the spinal fusion. The movement of the anatomical element 312B may be captured by the imaging devices 112 that output the captured image information depicting movement of the tracking marker 304B, and/or by measurements generated by the movement of the inertial sensor 308B. In some embodiments, the image information may be used by the navigation system 118 (using, for example, image processing 120 and/or segmentation 122) to determine a translational movement of the anatomical element 312B relative to the anatomical element 312A. Similarly, the navigation system 118 may, using one or more transformations 124 processing one or more measurements or readings generated by the inertial sensor 308B, determine a rotational movement of the anatomical element 312B relative to the anatomical element 312A. Using the determined movement of the anatomical element 312B, the navigation system 118 may be able to further update the registration of the anatomical element 312B to the surgical tool and/or adjust the surgical plan based on the movement of the anatomical element 312B.

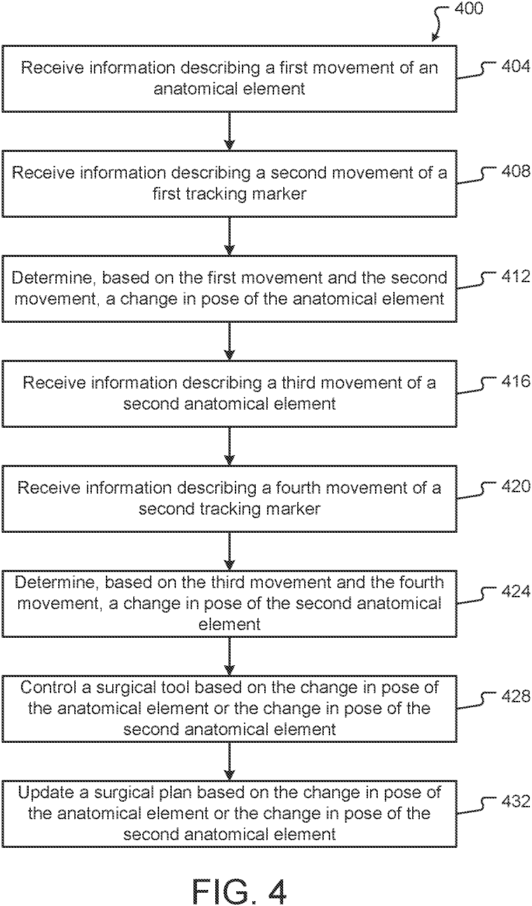

FIG. 4 depicts a method 400 that may be used, for example, to determine a movement of an anatomical element and adjust or update the surgery or surgical procedure based on the determined movement.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as an image processing 120, a segmentation 122, a transformation 124, and/or a registration 128.

The method 400 comprises receiving information describing a first movement of an anatomical element (step 404). The anatomical element may be an anatomical element similar to or the same as the anatomical element 204, the anatomical element 312A, and/or the anatomical element 312B. The first movement may be similar to or the same as the movement 316. In some embodiments, the first movement may be a translational movement, a rotational movement, combinations thereof, and/or the like. In some embodiments, the received information may be or comprise measurements generated by the movement of one or more inertial sensors (e.g., inertial sensors 142). In some embodiments, the one or more inertial sensors may be disposed on the anatomical element, a known distance from the anatomical element, disposed inside one or more optical spheres or other tracking markers or devices (e.g., tracking markers 138) that are disposed on or near the anatomical element, combinations thereof, and/or the like. In some embodiments, the one or more inertial sensors may be similar to or the same as the inertial sensors 142, the inertial sensor 308A, and/or the inertial sensor 308B.

In one embodiment, the one or more inertial sensors may be disposed on one or more other anatomical elements proximate the anatomical element in addition to or alternatively to the one or more inertial sensors disposed on the anatomical element. For instance, the inertial sensors may be disposed on multiple vertebrae of the spine. In such embodiments, the first movement may be a movement of a vertebra relative to one or more other vertebra, with the first movement being captured by the one or more inertial sensors. In at least one embodiment, the one or more inertial sensors may be disposed on every vertebrae of the spine.

In some embodiments, the received information may include measurements or readings generated by the inertial sensors as the anatomical element moves. As the anatomical element moves, the anatomical element may bump, vibrate, or otherwise move other anatomical elements (e.g., a movement of a first vertebra causes a second vertebra to move), with such movement being captured by the one or more inertial sensors (such as sensors attached to or placed near to the other anatomical elements). In some embodiments, the measurements or readings may reflect a rotational movement of the anatomical element. For instance, the measurements or readings may be generated by one or more IMUs that capture the rotational movement of the anatomical element. The receiving information may in some instances contain information related to the measurements or readings of some or all of the inertial sensors used in the surgery or surgical procedure.

The step 404 may use one or more transformations (e.g., transformations 124) that receive the measurements or readings of the one or more inertial sensors and determine the first movement of the anatomical element. The transformations may be or comprise one or more models (e.g., machine learning models, neural networks, etc.) that predict or estimate the movement of the anatomical element. In other embodiments, the transformations may transform the measurements or readings of one or more IMUs that reflect a rotational movement of the anatomical element. For instance, the transformations may take three separate readings in each direction in 3D generated by the IMUs transform the individual coordinate readings into an overall first movement in 3D space.

The method 400 also comprises receiving information describing a second movement of a first tracking marker (step 408). The first tracking marker may be similar to or the same as the tracking markers 138, the tracking marker 304A, and/or the tracking marker 304B. For instance, the first tracking marker may be an optical sphere attached to or otherwise disposed on the anatomical element.

The second movement of the first tracking marker may be captured by one or more imaging devices (e.g., imaging devices 112), with image data relating to the second movement sent to a navigation system, such as the navigation system 118. For instance, the imaging devices 112 may provide one or more images (or other image data related to the movement) that depict the tracking marker moving from a first position to a second position. The navigation system 118 may process the captured images (e.g., using image processing 120) to determine the movement and the resulting second position of the first tracking marker. The image processing 120 may be or comprise models, filters, algorithms, combinations thereof, and/or the like that receive the image data and output the determined movement and/or the second position of the first tracking marker. In some embodiments, the image data may be processed using one or more segmentations 122 before being processed by the image processing 120. The segmentation 122 may segment the images into discrete sections and/or identify one or more components depicted in the images or image data (such as anatomical elements, tracking markers, etc., and/or combinations thereof). Such segmentation may ease the computational requirements of the image processing by facilitating the identification of the first tracking marker in the image data.

The image processing 120 may use the identified components in determining the second movement of the first tracking marker. The image processing 120 may receive or access (e.g., from a database) coordinates associated with the first tracking marker (e.g., coordinates of the first tracking marker in a known coordinate space such as a patient coordinate space based on when the first tracking marker was attached to the anatomical element preoperatively) and may determine the second position of the first tracking marker after the second movement has occurred. The image processing 120 may determine the second position by, for example, mapping changes in pixel values into corresponding changes in coordinates in a known coordinate space. In some embodiments, the image processing 120 may take into account the second position of the first tracking marker relative to other tracking markers depicted in the images or image data and/or relative to a fixed point in space known to the navigation system 118 (e.g., a fixed reference point or marker depicted in the images or image data). The navigation system 118 may use the difference in coordinate values between the first position and the second position of the first tracking marker to determine the second movement of the first tracking marker. In such embodiments, the navigation system 118 may use one or more transformations (e.g., transformations 124) to transform the second movement of the first tracking marker into corresponding movement of the anatomical element based on, for example, a known physical relationship between the first tracking marker and the anatomical element (such as when the first tracking marker is disposed on the anatomical element) as discussed further in step 412.

In some embodiments, the image processing 120 may use one or more models to predict the change in pose. The models may be machine learning models, neural networks, and/or the like that receive the images or image data related to the first tracking marker and determine the movement of the first tracking marker. The models may be trained on historic data related to similar movements occurring during similar surgeries or surgical procedures. For example, a model trained on data related to spinal movements during a spinal fusion may be used in determining the second movement of a first tracking marker attached to a vertebra during a spinal fusion. In some embodiments, the models may output predicted or estimated coordinates based on the second movement of the first tracking marker.

The method 400 also comprises determining, based on the first movement and the second movement, a change in pose of the anatomical element (step 412). The step 412 may make use of one or more transformations (e.g., transformations 124) in determining the change in pose of the anatomical element. The transformations may use the first movement in determining a rotational movement of the anatomical element and use the second movement in determining a translational movement of the anatomical element. For example, the first movement may be a rotation of the anatomical element about a first axis, while the second movement may be a translational movement in a first direction. Using such information, the transformation may output a final pose based applying the first rotation about the first axis and the translational movement in the first direction to the known coordinates of the anatomical element and output the resulting coordinates. In some embodiments, the transformations may be machine learning models or other models trained on historical data of similar movements of similar anatomical elements (e.g., vertebrae undergoing spinal fusion surgery) that take the first movement and the second movement and predict the final pose of the anatomical element.

In some embodiments, the first movement of the anatomical element may be based only on rotational data provided by the one or more inertial sensors. Additionally or alternatively, the second movement of the first tracking marker may be based only on the translational movement of the first tracking marker. In such embodiments, the data directed toward the translational data provided by the one or more inertial sensors and/or the data directed toward the rotational data provided or determined by the first tracking marker may be discarded or otherwise not used in the final change in pose calculation.

The method 400 also comprises receiving information describing a third movement of a second anatomical element (step 416). In some embodiments, the step 416 may be similar to or the same as the step 404. For instance, the second anatomical element may be a vertebra proximate to the anatomical element (which may also be a vertebra), with the received information describing the third movement including measurements or readings from one or more inertial sensors disposed on or near the second anatomical element. In some embodiments, the third movement of the second anatomical element may be caused by the movement of the first anatomical element, such as when the second anatomical element is directly or indirectly affected by movement of the first anatomical element (e.g., the first and second anatomical elements are vertebrae that abut or contact one another, the anatomical elements are vertebrae linked with rods threaded through pedicle screws).

The method 400 also comprises receiving information describing a fourth movement of a second tracking marker (step 420). In some embodiments, the step 420 may be similar to or the same as the step 408. For instance, the fourth movement may be the movement of a second tracking marker that may be tracked by an imaging device. The images or image data captured by the imaging device may be used by the navigation system to determine the fourth movement. In some embodiments, the fourth movement of the second tracking marker may be directly or indirectly caused by the movement of the first anatomical element.

The method 400 also comprises determining, based on the third movement and the fourth movement, a change in pose of the second anatomical element (step 424). In some embodiments, the step 424 may be similar to or the same as the step 412. In some embodiments, the change in pose of the second anatomical element may be compared with the determined change in pose of the first anatomical element of the step 412 to, for example, verify the accuracy of the determined change in pose. For instance, the second anatomical element may abut the first anatomical element or be otherwise disposed proximate the first anatomical element. As such, movement of the first anatomical element may directly or indirectly cause the movement of the second anatomical element. The step 412 may use such a relationship (which may be reflected in data from a surgical plan, data stored in a database, etc.) to verify the accuracy the determined change in pose. As an example, if the first anatomical element and the second anatomical element are physically connected, and the first anatomical element is determined as having moved in a first direction, a determined change in pose of the second anatomical element that does not have a movement in the first direction may indicate that one or more of the determined changes in pose may be inaccurate. In such embodiments, the inconsistencies in the pose changes may cause the method 400 to return to steps 420 and/or 416 to re-evaluate the movement of the second anatomical element and/or the second tracking marker attached thereto. In such re-evaluations, the steps 420 and/or 416 may use different models and/or algorithms as those previously used, or may use the same models and/or algorithms subject to changes in various parameters (e.g., the steps may use the same models with different thresholding parameters).

The method 400 also comprises controlling a surgical tool based on the change in pose of the anatomical element or the change in pose of the second anatomical element (step 428). The surgical tool may be a drill, reamer, cutter, ablator, or the like that may operate on the first anatomical element and/or the second anatomical element. The surgical tool may be held by or otherwise attached to a robotic arm (e.g., robotic arm 116) that controls the pose (e.g., position and/or orientation) of the surgical tool during a surgery or surgical procedure. For instance, the surgery may be a spinal fusion, and the surgical tool may be a drill that is moved by the robotic arm to a surgical site. The drill may be maneuvered by the robotic arm to drill into a vertebra (e.g., for the purposes of inserting a pedicle screw). During the course of the surgery or surgical procedure, the surgical tool may be tracked by the navigation system using one or more imaging devices. In some embodiments, the step 428 may cause the robotic arm to move such that the pose of the surgical tool changes based at least in part on the change in pose of the first anatomical element and/or the second anatomical element.

For instance, the surgical tool may drill into the first anatomical element (e.g., a first vertebra) such that the surgical tool generates an incidental force on the second anatomical element (e.g., a second vertebra abutting the first vertebra), which causes the second anatomical element to move. The movement of the second anatomical element may comprise a translational and/or rotational movement that are captured by measurements from one or more inertial sensors and image data generated from imaging devices tracking one or more tracking markers. Such movement may be used to determine a change in pose of the second anatomical element from an initial pose to a new pose. The use of the surgical tool may be changed or altered based on the new pose. For example, the power provided to the surgical tool may be reduced so as to prevent or reduce the probability of further movement of the second anatomical element.

In some embodiments, the step 428 may include updating a registration of the surgical tool and/or the robotic arm to one or more anatomical elements (including, for example, the first anatomical element and/or the second anatomical element). The movement of the one or more anatomical elements may affect the accuracy of the pose of the surgical tool relative to one or more anatomical elements. For instance, the surgical tool may drill into the first anatomical element, but vibrations generated as the surgical tool operates may create a force that moves or displaces the second anatomical element from its initial pose. After the movement of the second anatomical element, the surgical tool may no longer be registered with the second anatomical element. In other words, the surgical tool may no longer be able to drill into the second anatomical element at the planned location and/or at the planned angle because the second anatomical element is no longer located at the same coordinates that were used to register the surgical tool to the second anatomical element.

The step 428 may update the registration using one or more registrations 128. The registrations 128 may be or comprise algorithms and/or models capable of receiving the new coordinates of an anatomical element (e.g., the first anatomical element, the second anatomical element, combinations thereof, and/or the like) and mapping the coordinates into a coordinate space compatible with or shared with the surgical tool (e.g., a surgical tool coordinate space, a coordinate space shared by the surgical tool and the anatomical element, a patient coordinate space, etc.). The updated registration may be used to manipulate the robotic arm to position the surgical tool relative to the anatomical element. In such embodiments, the surgical tool may be placed in the same pose relative to the anatomical element as what was originally planned, albeit in different coordinates in a commonly shared coordinate space.

The method 400 also comprises updating a surgical plan based on the change in pose of the anatomical element or the change in pose of the second anatomical element (step 432). The surgical plan may be retrieved (e.g., from a database) and updated based on the change in pose of the first and/or the second anatomical element. In some embodiments, the updating may include revising or adding in the new coordinates associated with the anatomical element after the pose of the anatomical element has changed. Additionally or alternatively, the details of the surgery or surgical procedure may be changed or updated based on the change in pose. The surgical plan may be updated such that the surgical tool operates on a different portion of an anatomical element. As an example, the surgical plan may initially detail drilling into a first portion of the first anatomical element at a first angle and into the second anatomical element at a second angle. The drilling of the first anatomical element may create the change in pose of the second anatomical element, which is captured and determined by, for example, the navigation system. Since the second anatomical element has moved, the surgical plan may be updated such that the surgical tool is to drill into the second anatomical element at a third angle different from the second angle. The change in angle may be determined based on surgical consequences of the movement of the second anatomical element (e.g., drilling into the second anatomical element at the second angle when the second anatomical element has moved would cause damage, result in surgical inefficiencies, or otherwise be undesirable). The extent of changes to the surgical plan are no way limited, and examples include changing the type of surgical tool used to perform the surgery or surgical procedure (e.g., using a reamer instead of a drill), changing the operating parameters of the surgical tool (e.g., power, torque, tool bit sizes), changing how the surgical tool performs the operation on the anatomical element (e.g., angle, depth, speed, etc. of the drilling, cutting, sawing, reaming, etc.), changing planned movement and/or navigation paths of the robotic arm to position the surgical tool, combinations thereof, and/or the like.

The present disclosure encompasses embodiments of the method 400 that comprise more or fewer steps than those described above, and/or one or more steps that are different than the steps described above.

As noted above, the present disclosure encompasses methods with fewer than all of the steps identified in FIG. 4 (and the corresponding description of the method 400), as well as methods that include additional steps beyond those identified in FIG. 4 (and the corresponding description of the method 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system, comprising:
a processor; and
a memory storing data thereon that, when executed by the processor, cause the processor to:
receive, from an inertial sensor disposed proximate an anatomical element, a reading indicative of a first movement of the anatomical element;
determine a second movement of a fiducial marker being positioned with a known physical relationship to the inertial sensor; and
determine, based on the first movement and the second movement, a change in pose of the anatomical element.

2. The system of claim 1, wherein the inertial sensor comprises an inertial measurement sensor (IMU) disposed on the anatomical element.

3. The system of claim 2, wherein the fiducial marker is disposed on the IMU.

4. The system of claim 1, wherein the anatomical element comprises a vertebra.

5. The system of claim 1, wherein the inertial sensor comprises at least one of a gyroscope or an accelerometer.

6. The system of claim 1, wherein the fiducial marker comprises an optical sphere.

7. The system of claim 1, wherein the first movement comprises a translational movement, wherein the second movement comprises a rotational movement about a first axis associated with the anatomical element, and wherein the change in pose of the anatomical element is determined to include at least some of the translational movement and at least some of the rotational movement about the first axis.

8. The system of claim 1, wherein an imaging device captures the second movement of the fiducial marker.

9. The system of claim 1, wherein the data further cause the processor to:
maneuver a robotic arm to move based on the determined change in pose of the anatomical element.

10. A method for tracking a movement of an anatomical element, the method comprising:
receiving, from a first inertial sensor attached to the anatomical element, first information indicative of the movement of the anatomical element;
receiving, from a first imaging device, second information indicative of a second movement of a first tracking marker; and
determining, based on the first information and the second information, a change in pose of the anatomical element.

11. The method of claim 10, further comprising:
receiving, from a second inertial sensor attached to a second anatomical element, third information indicative of a third movement of the second anatomical element;
receiving, from a second tracking marker, fourth information indicative of a fourth movement of a second tracking marker; and
determining, based on the third information and the fourth information, a change in pose of the second anatomical element.

12. The method of claim 11, further comprising:
controlling a surgical tool based on at least one of the change in pose of the anatomical element or the change in pose of the second anatomical element.

13. The method of claim 11, further comprising:
updating, based on at least one of the change in pose of the anatomical element or the change in pose of the second anatomical element, a surgical plan.

14. The method of claim 10, wherein the first tracking marker is an optical sphere.

15. The method of claim 14, wherein the optical sphere is tracked by the first imaging device.

16. The method of claim 10, wherein the first tracking marker comprises an Infrared Light Emitting Diode (IRED).

17. The method of claim 10, further comprising:
registering a robotic arm to the anatomical element.

18. The method of claim 10, wherein the movement is a translational movement, and wherein the second movement is a rotational movement about a first axis of the anatomical element.

19. A system, comprising:
an imaging device;
an inertial measurement unit (IMU) disposed on an anatomical element;
at least one fiducial marker disposed on or in proximity to the IMU;
a processor; and
a memory storing data thereon that, when processed by the processor, cause the processor to:
receive, from the IMU, information describing a translational movement of the anatomical element;
receive, from the imaging device, information describing a rotational movement of the at least one fiducial marker; and
determine, based on the information describing the translational movement and the information describing the rotational movement, a pose of the anatomical element.

20. The system of claim 19, wherein the fiducial marker comprises an optical sphere, and wherein the IMU comprises at least one of an accelerometer or a gyroscope.
